(19) United States
(12) Patent Application Publication
(10) Pub. No.: US 2007/0014789 A1
(43) Pub. Date: Jan. 18, 2007

(54) Humanized antibody conjugates and related methods, assays, reagents, and kits

(75) Inventors: Linda Marie Anderson-Mauser, Elkhart, IN (US); Robert John Dwyer, New Fairfield, CT (US); Mary Margaret Foltz, Cassopolis, MI (US)

Correspondence Address: BAYER HEALTHCARE, LLC;LAW & PATENTS DEPT, 511 BENEDICT AVENUE, TARRYTOWN, NY, 10591-5097, US

(73) Assignee: Bayer HealthCare LLC, Tarrytown, NY (US)

(21) Appl. No.: 11/457,537

(22) Filed: Jul. 14, 2006

Related U.S. Application Data
(60) Provisional application No. 60/699,618, filed on Jul. 15, 2005.

(51) Int. Cl. A61K 39/395; C07K 16/44

(52) U.S. Cl. 424/133.1; 530/388.15

(57) ABSTRACT

The invention provides synthetically and recombinantly-derived humanized antibody conjugates and related methods, diagnostic assays, reagents, and kits. In one embodiment, the invention provides humanized antibody conjugates comprising a human immunoglobulin fragment which is bound by a cross-linking functional group to a non-human antibody fragment (e.g., a non-human monoclonal antibody fragment) comprising an antigen-binding amino acid sequence.

### CROSS REFERENCE TO RELATED APPLICATIONS

This application claims the priority of U.S. Provisional Application No. 60/699618, filed Jul. 15, 2005, and U.S. Provisional Application No. 60/708361, filed Aug. 31, 2005, both of which are hereby incorporated herein by reference.

### FIELD OF THE INVENTION

The invention relates to synthetically and recombinantly-derived humanized antibody conjugates and related methods, analytical assays including, for example, diagnostic assays, reagents, and kits.

### BACKGROUND OF THE INVENTION

Analytical assays, particularly diagnostic assays, use as a positive control reagent sera samples taken from a donor who has experienced an immune reaction caused by exposure to the antigen targeted by the assay. It is often very difficult and expensive to obtain serum-based control reagents for assays which detect antibodies to certain antigens. For particular grass pollens and hepatitis viral strains, identifying willing human donors who have a suitable serum antibody titer and epitope specificity can entail a worldwide search. Using sera-derived control reagents also introduces complexities associated with batch-to-batch and donor-to-donor variability.

Recombinant humanized monoclonal antibodies (“Mabs”) can be designed both to target a specific epitope and to contain human immunoglobulin amino acid sequences which are recognized by assay reagents. In humanized, murine-derived Mabs, the antibody comprises around five percent or less of murine protein. See Presta, et al., J. Immunol. 1993 Sep. 1; 151(5):2623-32.

References which describe the therapeutic use of recombinant, humanized Mabs are legion. For example, Borchmann, et al. Blood, Vol. 100; p. 3101, describes the production of a bifunctional Fab2 by combining Fab′ fragments from two different humanized antibodies. U.S. Pat. No. 6,699,974 describes the recombinant generation of reshaped human anti-HM 1.24 antibody comprising human and murine antibody chains.

Notwithstanding their epitopic specificity, recombinant, humanized Mabs have not been used as diagnostic assay control reagents. U.S. Pat. No. 6,680,209 discloses detecting an analyte in a human sample containing human antibodies that specifically bind to antibodies from a nonhuman species, but does not describe the use of recombinant humanized antibodies as control reagents. Biotechniques 35: 672-674 (Oct. 2003) describes high-throughput isolation of recombinant antibodies against recombinant allergens, but does not describe the use of recombinant humanized antibodies as control reagents.

Accordingly, there is a need for affordable, effective control reagents which do not exhibit batch-to-batch and donor-to-donor variability and which can be used in a wide variety of diagnostic assays.

### SUMMARY OF THE INVENTION

The invention provides humanized antibody conjugates comprising a human immunoglobulin fragment which is bound by a cross-linking functional group to a non-human antibody fragment (e.g., a non-human monoclonal antibody fragment) comprising an antigen-binding amino acid sequence. Humanized antibody conjugates of the invention are chemically synthesized to exhibit antigen binding specificities which are recognized by reagents used in a given diagnostic assay.

Unlike known sera-derived diagnostic assay control reagents, humanized antibody conjugates of the invention are robust compositions which do not exhibit batch-to-batch or donor-to-donor variation. Further, humanized antibody conjugates of the invention can be made inexpensively and quickly and prove well-suited for use as control reagents or capture antibodies in competitive and sandwich immunoassays.

In one embodiment, humanized antibody conjugates of the invention comprise a human antibody fragment (e.g., a Fc fragment of either human IgG or human IgE) bound by a cross-linking functional group to an antibody fragment (e.g., a Fab2 fragment) which is derived from a non-human antibody (e.g., a non-human Mab) and which contains an antigen-binding amino acid sequence. The antigen may comprise, for example, an allergen, a virus, a bacteria, or a ligand associated with a disease.

In a preferred embodiment, humanized antibody conjugates of the invention comprise a Fc fragment of human IgG bound by a cross-linking functional group to a Fab2 fragment which is derived from a murine Mab and which contains an antigen-binding amino acid sequence.

In another preferred embodiment, the invention provides humanized antibody conjugates in which: (1) an antibody fragment (e.g., a Fab2 fragment) which is derived from a non-human antibody and which contains an antigen-binding amino acid sequence is bound by a cross-linking functional group to a human antibody fragment (e.g., a Fc fragment of either human IgG or human IgE); and (2) the cross-linking functional group is formed by the reaction of: (a) a thiol (disulfide)-binding moiety which has been derivatized onto the human antibody fragment; and (b) a reduced thiol (disulfide) bond on the non-human antibody fragment.

In still another preferred embodiment, the invention provides humanized antibody conjugates in which a murine Mab Fab2 fragment which contains an antigen-binding amino acid sequence is bound by a cross-linking functional group to a Fc fragment of human IgG, and wherein (1) the cross-linking functional group is formed by the reaction of: (a) a thiol (disulfide)-binding moiety which has been derivatized onto the Fc fragment by reaction of the Fc fragment with a hetero-bifunctional cross-linking agent; and (b) a reduced thiol (disulfide) bond on the Fab2 fragment; and (2) the Fab2 fragment is obtained by an aspartic proteinase digestion of the murine Mab which preserves the thiol (disulfide) bond on the Fab2 fragment.

The invention also provides methods for synthesizing humanized antibody conjugates chemically, as well diagnostic assays, methods, reagents, and kits which use such chemically synthesized humanized antibody conjugates.

In another embodiment, the invention provides diagnostic methods and kits which use recombinant, humanized Mabs as control reagents.

These and other aspects of the invention are described further in the following detailed description.

### BRIEF DESCRIPTION OF THE FIGURES

FIG. 1 illustrates Novex electrophoresis gel analyses of humanized antibody conjugate Fab2 fragments, as determined in accordance with the experiment(s) of Example 1.

FIG. 2 illustrates Novex electrophoresis gel analysis of human IgG digested with ficin, as determined in accordance with the experiment(s) of Example 1.

FIG. 3 illustrates SDS polyacrylamide gel analysis of the digested humanized antibody conjugate Fc fragment, as determined in accordance with the experiment(s) of Example 1.

FIG. 4 illustrates the elution profile of the humanized NS5 conjugation reaction mixture from a chromatography column.

FIG. 5 illustrates SDS gradient gel analysis of humanized hepatitis C antibody conjugate fractions, as determined in accordance with the experiment(s) of Example 1 and illustrated in FIG. 4.

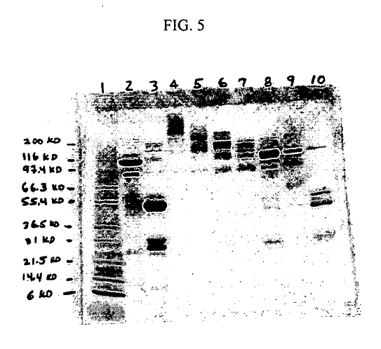

### DETAILED DESCRIPTION OF THE INVENTION

1. Definitions.

The following definitions apply unless otherwise noted.

An “allergen” means any substance that triggers an immune response.

An “analytical method” shall mean the detection, measurement, determination, characterization or other assessment of a substance or substances in, or the composition, state or other condition of, a test sample, including, without limitation, (a) testing performed for research or commercial purposes, such as medical, veterinary or industrial testing, and (b) in vitro or in vivo testing where the test sample is of human or animal origin, and including, without limitation, (a) qualitative and quantitative assays, (b) multiplexing assays, classification, sequencing and other characterization assays, (c) indexing, reflex and combination assays involving the detection, measurement, determination, characterization or other assessment of more than one substance in, or condition of, a test sample, and/or the calculation of an index or application of a mathematical or other algorithm, and (d) the performance of calibration, control, or other standardization steps. The term analytical method as used in the description of the present invention includes those methods, reagents, kits, calibrators, controls that are used for the testing of human or animal specimens for the purpose of diagnosis, prognosis, or monitoring the progress of disease or monitoring the effect of treatment of disease, in the human or animal from which the specimens were taken.

An “antibody” is an intact immunoglobulin molecule comprising two of each of immunoglobulin light and heavy chains. Unless otherwise noted, “antibody” includes polyclonal or monoclonal antibodies. Antibodies used in the invention can be prepared by techniques generally known in the art, and are typically generated as either an isolated, naturally occurring protein, as a recombinantly expressed protein, or as a synthetic peptide representing an antigenic portion of a natural protein.

The light chains of antibodies (immunoglobulins) from any vertebrate species can be assigned to one of two clearly distinct types, called kappa (κ) or lambda (λ) based on the amino sequences of their constant domain. Depending on the amino acid sequences of the constant domain of their heavy chains, antibodies can also be assigned to different classes. There are at least five (5) major classes of antibodies: IgA, IgD, IgE, IgG and IgM, and several of these may be further divided into subclasses (isotypes), e.g IgG-1, IgG-2, IgG-3 and IgG4; IgA-1 and IgA-2.

An “antibody-binding region” maintains the structure of the antibody that interacts with the antigen. Generally, the combined light and heavy variable domains of an antibody constitute the antibody-binding region.

The term “antibody fragment” refers to a portion of a full-length antibody, and generally refers to the antigen binding or variable region. Examples of antibody fragments include the Fc, Fab, Fab′, F(ab′)₂ (also referred to herein as the Fab2 fragment), and Fv fragments. Papain digestion of antibodies produces two identical antigen binding fragments: (1) the Fab fragment, which has a single antigen binding site; and (2) a residual “Fc” fragment, which crystallizes readily. Pepsin digestion yields:

an F(ab′)₂ fragment that has two antigen binding fragments which are capable of cross-linking antigens; and (2) a residual fragment pFc′.

An “Fv” fragment is the minimum antibody fragment which contains a complete antigen recognition and binding site. This region consists of a dimer of one heavy and one light chain variable domain in a tight, non-covalent association (V_{H}-V_{L} dimer). It is in this configuration that the three complementarity determining regions (CDRs) of each variable domain interact to define an antigen binding site on the surface of the V_{H}-V_{L} dimer. Collectively, the six CDRs confer antigen binding specificity to the antibody. However, even a single variable domain (or half of an Fv comprising only three CDRs specific for an antigen) has the ability to recognize and bind antigen, although at a lower affinity than the entire binding site.

The Fab fragment also contains the constant domain of the light chain and the first constant domain (CH1) of the heavy chain. Fab′ fragments differ from Fab fragments by the addition of a few residues at the carboxyl terminus of the heavy chain CH1 domain including one or more cysteines from the antibody hinge region. Fab′-SH or Fab′ fragments contain cysteine residue(s) of the constant domains and have a free thiol group.

Typically, F(ab′) fragments are produced by cleavage of the disulfide bond at the hinge cysteines of the F(ab′)₂ pepsin digestion product.

A “chimeric” antibody is an antibody homolog in which all or part of the hinge and constant regions of an immunoglobulin light chain, heavy chain, or both, have been substituted for the corresponding regions from another immunoglobulin light chain or heavy chain. For example, “chimeric antibody” refers to an antibody in which the variable region is derived from a nonhuman antibody, e.g., a murine or rat antibody, and the constant region is derived from a human antibody.

The technology for producing monoclonal antibodies is well known to those of ordinary skill in the art. An immortal cell line (typically myeloma cells) is fused to lymphocytes (typically splenocytes) from a mammal immunized with whole cells expressing a given antigen and the culture supernatants of the resulting hybridoma cells are screened for antibodies against the antigen. See, e.g., Kohler et al., 1975, Nature 265: 295-497; Campbell, Monoclonal Antibody Technology. Laboratory Techniques in Biochemistry and Molecular Biology (Elsevier Netherlands 1984); St. Groh et al., J. Immunol. Methods, 35, pp. 1-21 (1980);; and Coligan et al. (eds.), Current Protocols in Immunology, Vol. 1, pages 2.5.1-2.6.7 (John Wiley & Sons 1991).

Humanized monoclonal antibody homologs can be prepared, e.g., by using in vitro-primed human splenocytes. See Boerner et al., 1991, J. Immunol. 147:86-95. Humanized monoclonal antibodies can also be prepared by repertoire cloning. See Persson et al., 1991, Proc. Nat. Acad. Sci. USA 88: 2432-2436; Huang, et al. 1991,J. Immunol. Methods 141: 227-236.

Monoclonal antibodies can be isolated and purified from hybridoma cultures by a variety of well-established techniques. Such isolation techniques include affinity chromatography with Protein-A Sepharose, size-exclusion chromatography, and ion-exchange chromatography. See, e.g., Coligan, supra at pages 2.7.1-2.7.12 and 2.9.1-2.9.3. See also Baines et al., Methods in Molecular Biology, Vol. 10, pp. 79-104 (The Humana Press, Inc. 1992).

A “control reagent” is a solution which has a known concentration of an analyte (e.g., antibody) and which, when used in a serology immunoassay, provides a positive control which validates assay function.

“Cross-linking functional groups” form molecular bridges that link functional groups of two different molecules. Cross-linking functional groups contain two reactive groups, one of which usually reacts with a primary amine group (e.g., N-hydroxy succinimide) and the other of which usually reacts with a thiol group (e.g., pyridyl disulfide, maleimides, halogens, etc.). Through a thiol reactive group, a cross-linker bound to a first protein can react with a cysteine residue (free sulflhydryl group) of another protein.

To form a cross-linking functional group which will bind two antibody fragments in accordance with the invention, one antibody fragment may be derivatized with a cross-linking moiety through reaction with a hetero-bifunctional cross-linking agent, and an intrinsic cross-linker such as a thiol (disulfide) bond on the other fragment may be reduced for reaction with the cross-linking moiety by exposure to a reducing agent.

For example, reaction of a human Fc fragment with hetero-bifunctional cross-linking agent will derivatize the human Fc fragment by adding a thiol (disulfide)-binding group (e.g., a maleimide group) to primary amines accessible on the protein surface. Reaction of a non-human Fab2 fragment with reducing agents will reduce Fab2 fragment disulfide bonds and the reduced Fab2 fragment disulfide bonds will bind to the Fc fragment thiol (disulfide)-binding group to form a cross-linking functional group which will bind the two antibody fragments.

“Hetero-bifunctional cross-linking agents” include but are not limited to succinimidyl-4-(N maleimidomethyl) cyclohexane-1-carboxylate (“SMCC”); 4-(N maleimidomethyl)cyclohexane-1-carboxylic 3-sulfo-n-hydroxysuccinimide ester (“sulfo-SMCC”); m-maleimidobenzoyl-N-hydroxysulfosuccinimide ester (MBS); sulfo-MBS; N-succinimidyl-3-(2-pyridyl-thio) propionate (SPDP); N-succinimidyl(4-iodoacteyl) aminobenzoate (“SIAB”); succinimidyl-4-(p-maleimidophenyl)butyrate (“SMBP”); N-(y-maleimidobutyryloxy)succinimide ester (“GMBS”); and 4-(4-N-maleimidopohenyl) butyric acid hydrazide (“MPBH”).

“Reducing agents” include but are not limited to mercaptoethylamine (MEA); N-oxydiethylene-2-benzothiazolyl sulfonamide; tri-n-butylphosphine; dithiothreitol; and beta-mercaptoethanol (BME).

“Cysteine (thiol) proteinases” include but are not limited to papain, ficin, actinidin, bromelain, mammalian lysosomal cathepsins, the cytosolic calpains (calcium-activated), and parasitic proteases (e.g Trypanosoma, Schistosoma).

“Aspartic proteinases” include but are not limited to pepsin, chymosin, lysosomal cathepsins D, processing enzymes such as renin, and fungal proteases (e.g., penicillopepsin, rhizopuspepsin, endothiapepsin).

As used herein, an “epitope” is a molecular region on the surface of an antigen which is capable of eliciting an immune response and which is also capable of binding to or combining with the specific antibody produced by such a response.

“FR” is an abbreviation for Framework Region, which comprises the portions of the variable regions of an antibody which are adjacent to, or flank, the CDRs. In general, these regions have a structural function that affects the conformation of the variable region and are less directly responsible for the specific binding of antigen to antibody.

“Humanized antibodies” are antibody molecules that bind to a specific antigen and have one or more CDRs from a non-human species and a framework region from a human immunoglobulin molecule. See, e.g. U.S. Pat. No. 5,585,089; Riechmann et al., Nature, 332:323 (1988). In one example, murine or rat CDRs are transferred from heavy and light variable chains of mouse or rat antibodies into a variable region designed to contain a number of amino acid residues found within the FR region in human IgG. General techniques for cloning murine immunoglobulin variable domains are described in Orlandi et al., Proc. Nat'l Acad. Sci. USA 86:3833 (1989). Techniques for producing humanized monoclonal antibodies are described in Jones et al., Nature 321:522 (1986), Riechmann et al., supra; Verhoeyen et al., Science 239:1534 (1988); Carter et al., Proc. Nat'l Acad. Sci. USA 89:4285 (1992); Sandhu, Crit. Rev. Biotech., 12:437 (1992); and Singer et al., J. Immun. 150:2844 (1993). See also Boemer et al., 1991; Persson et al.; and Huang, et al, supra.

A “humanized recombinant antibody” is an antibody which is initially derived from a nonhuman mammal in which recombinant DNA technology has been used to substitute some or all of the amino acids not required for antigen binding with amino acids from corresponding regions of a human immunoglobulin light or heavy chain. References cited above disclose methods for synthesizing humanized recombinant antibody which are well-known to those of ordinary skill in the art.

“Humanized antibody conjugates” of the invention comprise a human immunoglobulin fragment which is bound by cross-linking functional groups to a ligand-binding region comprising a non-human antibody fragment, e.g., a non-human antibody fragment.

“Immunoassays” determine the presence of a patient diagnostic serum marker in a biological sample by reacting the sample with an antibody that binds to the serum marker, the reaction being carried out for a time and under conditions allowing the formation of an immunocomplex between the antibodies and the serum markers. The quantitative determination of such an immunocomplex is then performed.

In one version, the antibody used is an antibody generated by administering to a mammal (e.g., a rabbit, goat, mouse, pig, etc.) an immunogen that is a serum marker, an immunogenic fragment of a serum marker, or an anti-serum marker-binding idiotypic antibody. Other useful immunoassays feature the use of serum marker-binding antibodies generally (regardless of whether they are raised to one of the immunogens described above). A sandwich immunoassay format may be employed which uses a second antibody that also binds to a serum marker, one of the two antibodies being immobilized and the other being labeled.

Preferred immunoassays detect an immobilized complex between a serum marker and a serum marker-binding antibody using a second antibody that is labeled and binds to the first antibody. Alternatively, the first version features a sandwich format in which the second antibody also binds a serum marker. In the sandwich immunoassay procedures, a serum marker-binding antibody can be a capture antibody attached to an insoluble material and the second a serum marker-binding antibody can be a labeling antibody.

The assays used in the invention can be used to determine a blood marker, e.g., a plasma or serum marker in samples including urine, plasma, serum, peritoneal fluid or lymphatic fluid. Immunoassay kits for detecting a serum marker can also be used, and comprise a serum marker-binding antibody and the means for determining binding of the antibody to a serum marker in a biological sample. In preferred embodiments, the kit includes one of the second antibodies or the competing antigens described above.

The terms “protein”, “polypeptide”, and “peptide” are used interchangeably herein to refer to a polymer of amino acids. A protein may include more than one polypeptide chain. A polypeptide can be expressed recombinantly or can be produced synthetically.

“Solid phase” means a non-aqueous matrix to which an antibody or humanized antibody conjugate can adhere. Examples of solid phases include those formed partially or entirely of glass (e.g. controlled pore glass), polysaccharides (e.g., agarose), polyacrylamides, polystyrene, polyvinyl alcohol and silicones. In certain embodiments, depending on the context, the solid phase can comprise the well of an assay plate; in others it is a purification column (e.g. an affinity chromatography column). This term also includes a discontinuous solid phase of discrete particles.

2. Humanized Antibody Conjugates.

In a preferred embodiment of the invention, a non-human Mab such as a murine or rat Mab which binds to an antigen of interest is digested in an aspartic proteinase such as pepsin to separate the Mab Fab2 fragment from the Mab intact IgG 1. The non-human Mab is preferably digested: (1) in a buffered reaction mixture comprising excess antibody (e.g., around fifty to around two hundred times more antibody than aspartic proteinase on a weight basis); (2) at a pH of between about 3 to about 6 (most preferably at a pH of from about 4 to about 5); and (3) at temperature of around 30° C. to around 40° C.

Aspartic proteinase digestion yields a Fab2 fragment which retains a thiol bond between the two cysteines present on each of the two heavy and light chain fragments. An aspartic proteinase such as pepsin cuts behind the disulfide bond in the immunoglobulin (see Essential Immunology, Ivan Roitt (W. Clowes and Sons, 3^{rd} ed., 1977)). Available thiol groups which are removed from the binding site, and which are proximate to the pepsin digestion site, are reduced gently with mercaptoethylamine.

Human IgG is digested enzymatically using a cysteine (thiol) proteinase such papain and ficin to generate the Fc fragment. The Fc fragment is derivatized with a thiol (disulfide)-linking maleimide group by reaction with a hetero-bifunctional cross-linking agent such as SMCC.

Humanized antibody conjugates are formed by the reaction at room temperature of an excess of Fab2 fragments with Fc fragments (preferably in a weight ratio of Fab2:Fc of around 1.5:1 to 5.0:1, more preferably around 2:1 to 3:1); cross-linking functional groups join the Fab2 and Fc fragments through reaction of the reduced Fab2 thiol (disulfide) group and the Fab maleimide moiety.

The aforementioned features of the invention are illustrated further in the following examples.

### EXAMPLE 1

### Hepatitis C Humanized Antibody Conjugate

### Production of the Fab2 Fragment

Anti-HepC NS5 Mab (Bayer Clone ML414-14A2.5A4 Anti HCV NS5), deposited under the Budapest Treaty on Jul. 14, 2005 with the American Type Culture Collection, 10801 University Boulevard, Manassas, Va. 20110-2209, ATCC Dep. No. PTA-6861, (hereinafter “NS5”) was used to generate the antigen binding portion of the conjugate. NS5 was digested with pepsin to produce a Fab2 fragment from intact IgG1 as follows.

First, digestion time was optimized. NS5 was placed into a 0.1 M citrate, pH 3.5 buffer using a NAP-5 column (Pharmacia). The amount of pepsin (Sigma) used to digest NS5 was approximately 1/30 of NS5 weight and the reaction was incubated at 37° C. Samples were removed at regular intervals and an optimal digestion time of 45 minutes was determined by examining the progress of the digestion on a Novex electrophoresis gel. The Fab2 fragment prepared from the functioning capture portion of NS5 weighed approximately 100 KD.

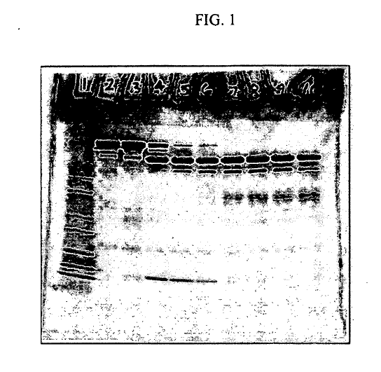

The Optimization of Pepsin Digestion of anti-HCV clone NS5 is illustrated in FIG. 1. Note that the Lanes correspond to the Samples as follows:

| Lane # | Sample |
|---|---|
| 1 | MW Std |
| 2 | undigested spI |
| 3 | t = 0 |
| 4 | t = 10 minutes |
| 5 | t = 20 minutes |
| 6 | t = 30 minutes |
| 7 | t = 45 minutes |
| 8 | t = 60 minutes |
| 9 | t = 1.5 hours |
| 10 | t = 2 hours |

To scale-up production of the Fab2 fragment, the digestion reaction was quenched with 1M Tris, and small digested fragments of the Fc region were thereafter removed by exhaustive dialysis of the reaction against PBS/2MM EDTA using a 30K MW membrane.

### Production of the Fc Fragment

Human IgG (Fitzgerald catalog #30-AI17) was digested with ficin to generate a humanized antibody conjugate Fc fragment. Since it was not known how the polyclonal human IgG preparation would tolerate digestion, experiments were conducted to identify the best way to prepare the Fc portion of the molecule.

Both papain and ficin enzymes (Sigma) were tested with a highly pure human IgG1 preparation. The antibody protein was exchanged to facilitate digestion into 50 mM Tris, 2 mM EDTA pH 7.0 buffer using NAP-5 (Pharmacia) columns. Both of the enzymes were tested at 1/30 (w/w) proportions relative to the antibody. Both enzyme and cysteine were added to the protein and digested at 37° C. At timed intervals, aliquots of the reaction were removed and the enzyme reaction stopped with n-ethylmaleimide. Aliquots were then examined on Novex gels for examination of the fragment sizes produced.

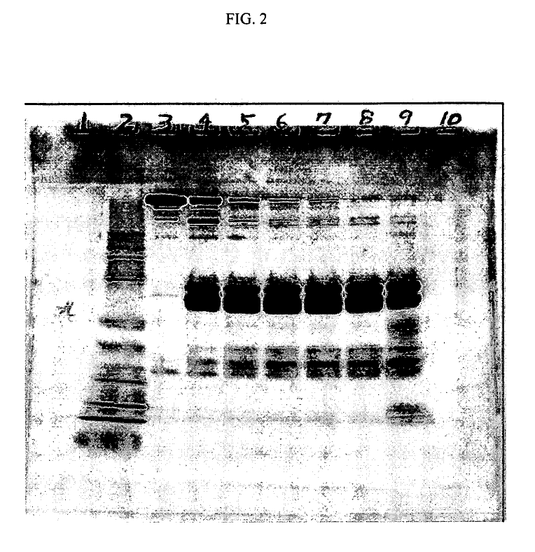

Since the Novex gels demonstrated that both ficin and papain produced identical results, the experiment was continued using only ficin. Longer incubation times with identical reaction conditions confirmed that 3.5 hrs was an optimal digestion period, although there remained a small portion of the heterogeneous polyclonal human IgG1 preparation that was resistant to digestion. The Optimization of Ficin Digestion of huIgG₁ Antibody is illustrated in FIG. 2. Note that the Lanes in FIG. 2 correspond to the Samples as follows:

| Lane # | Sample |
|---|---|
| 1 | empty |
| 2 | MW Marker |
| 3 | t = 0 |
| 4 | t = 1 hour |
| 5 | t = 2 hour |
| 6 | t = 2.5 hours |
| 7 | t = 3 hours |
| 8 | t = 3.5 hours |
| 9 | t = 4 hours |
| 10 | Empty |

The resultant Fc preparation was predominantly 50 KD in weight; no single molecular weight could be determined due to the heterogeneous nature of the native protein mixture.

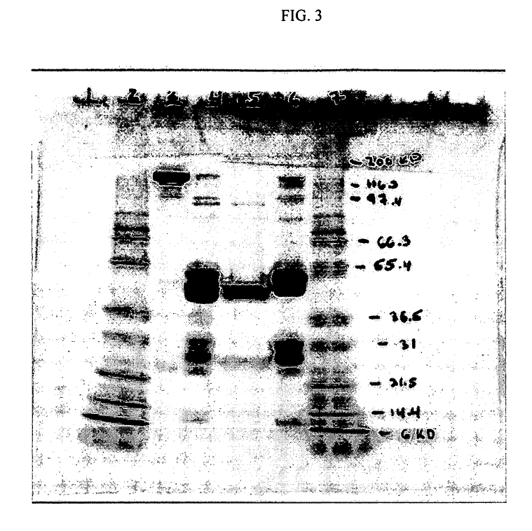

For the scale-up production of the Fc fragment, the digested, non-reactive medium was purified by passing it over a protein A column. This step served to remove the Fc fragment from the remaining digested portion of the IgG1 molecule by allowing the intact Fc to bind to the protein A resin, while allowing the remainder of the digest reaction to flow through the column. The intact Fc fragment was then eluted and collected. SDS polyacrylamide gel analysis demonstrated that the resultant Fc fragment pool was recovered. The SDS Gel at Various Steps in Isolation of Human Fc Fragment is illustrated in FIG. 3. Note that the Lanes in FIG. 3 correspond to the Samples as follows:

| Lane # | Sample |
|---|---|
| 1 | empty |
| 2 | MW Marker |
| 3 | undigested Ab |
| 4 | flow through from protein A column |
| 5 | isolated Fc fragment |
| 6 | MW Marker |
| 7 | empty |
| 8 | empty |
| 9 | empty |
| 10 | Empty. |

The Fab2 and Fc fragments were linked to form a humanized antibody conjugate as follows.

### Formation of the Humanized Antibody Conjugate

The Fab2 and Fc fragments obtained as described above were linked to form a humanized antibody conjugate as follows.

Because the Fab2 fragment was obtained by digestion of NS5 in pepsin as described above, the thiol (di-sulfide) bond between the two cysteines on the heavy and light chain fragments of NS5 was preserved. (Pepsin digestion cut the NS5 Fab2 fragment from intact IgG1 at a location which preserved a thiol (disulfide) bond between the Fab2 fragment heavy and light chains

This thiol (disulfide) bond on the Fab2 fragment was activated by reaction of the Fab2 fragment with 50 mM mercaptoethylamine (MEA) at approximately 37° C. for a period of around 90 minutes. Excess MEA was removed by desalting in a PBS 2 mM EDTA reaction buffer using a NAP25 column (Pharmacia).

The Fc fragment was derivatized by adding a maleimide group to primary amines accessible on the protein surface through reaction of the Fc fragment with SMCC. Specifically, Fc fragment was added to an approximately twenty-five fold molar excess of SMCC (Pierce) and the resultant reaction mixture was allowed to stand for 25 minutes at room temperature. Excess SMCC was quenched by the addition of glycine to the reaction mixture and excess SMCC was removed by desalting the protein mixture over a NAP25 column equilibrated in pH 7.4 PBS.

The Fab and Fc fragments were combined to form a reaction mixture comprising an approximately 2.4 fold molar excess of Fab to Fc and were allowed to react for approximately three hours at room temperature to form humanized Hep C antibody conjugate. Thereafter, the reaction mixture was stored overnight at 2° C.-8° C. and excess maleimide remaining in the reaction mixture was quenched by the addition of twelve-fold molar excess MEA.

Next, the humanized Fab:Fc conjugates were recovered using column chromatography. The MEA-quenched reaction mixture was chromatographed on a 1.6×60 Superdex 200 column (Pharmacia). A 7 milligram conjugate mixture in an approximately 1 mL volume was applied to the column and fractionated by size by elution in a PBS (pH 7.4) buffer at 0.2 ml/min to achieve optimal size separation.

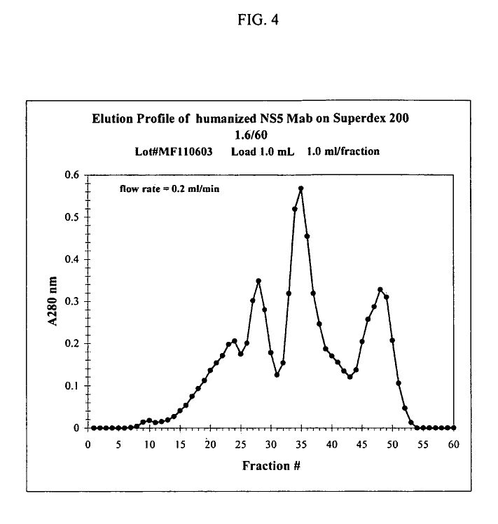

Fractions were collected and analyzed by SDS gradient gel (4-20%) for correct size. Although a single Fab′:Fc conjugate weighed approximately 100 KD, multimers containing more than one Fab per Fc resulted in peaks of greater than 100 KD. The elution profile of humanized NS5 Mab on Superdex 200 1.6 is illustrated in FIG. 4. (Lot# MF110603; Load 1.0 mL; 1.0 mL/fraction)

Pools of conjugate with varying molecular weights were obtained and used as a control reagent in the ADVIA Centaur® hepatitis C assay, as described in Example 2. (ADVIA Centaur® is a registered trademark of Bayer HealthCare LLC, Tarrytown, N.Y. U.S.A.)

### EXAMPLE 2

### Use of Humanized Hep C Antibody Conjugate as a Control Reagent

Humanized Hepatitis C antibody conjugates made in the experiment(s) of Example 1 were used as control reagents in diagnostic kits employed with the Bayer ADVIA Centaur® hepatitis C assay.

A sample was incubated with Solid Phase containing recombinant peptide HCV antigens. Antigen-antibody complexes will form if anti-HCV antibody is present in the sample. Lite Reagent containing monoclonal anti-human IgG labeled with acridinium ester was used to detect human anti-HCV IgG in the sample.

The system automatically performed the following steps: (1) it dispensed 10 uL of sample into a cuvette; (2) it dispensed 100 ul of a sample diluent and incubated the test sample for 5 minutes at 37° C.; (3) it dispensed 100 uL of Solid Phase reagent and incubated the reagent for 18 minutes at 37° C.; (4) it separated the Solid Phase from the mixture and aspirated the unbound reagent; (5) it washed the cuvette with Wash 1; (6) it dispensed 50 uL of Lite Reagent, (7) it incubated the mixture for 18 minutes at 37° C.; (8) it washed the cuvette with Wash 1; and (9) it dispensed 300 uL each of Acid Reagent and Base Reagent to initiate the chemiluminescent reaction.

A direct relationship exists between the level of anti-HCV present in the patient sample and the amount of relative light units (RLUs) detected by the system. The results are shown in the Table 1 below.

TABLE 1
Screening of Humanized NS5 monoclonal with HCV antigens

| | Relative Light Units | | |
|---|---|---|---|
| Sample | c200 | c22 | NS5 |
| Negative Control | 32045 | 28653 | 28718 |
| Positive Control | 457012 | 185861 | 36410 |
| anti-c200 | 317390 | 25762 | 19945 |
| anti-c22 | 32512 | 377491 | 10101 |
| MF110603-20* | 1705 | 3159 | 79612 |
| MF110603-24* | 1818 | 3261 | 76441 |
| MF110603-28* | 1684 | 3044 | 71191 |
| MF110603-35* | 1677 | 2880 | 3023 |
| MF110603-48* | 1852 | 3290 | 1777 |

*fractions tested at 10 ug/mL

In this experiment, patient samples containing antibodies either to HCV c200 or to HCV c22 antigens were used as controls. These patient samples specifically reacted to only c200 antigens and c22 antigens, respectively. Pooled fractions from Example I containing humanized anti-NS5 HCV antibodies reacted specifically to the recombinant NS5 antigen and not to the recombinant c200 or c22 antigens.

### EXAMPLE 3

### Allergen Assay

An assay which assists in the diagnosis of whether an individual is allergic to a particular antigen (e.g., pollen) requires a positive control to determine whether the assay is functioning properly and is being used correctly. This example describes how a humanized antibody conjugate of the invention can be used as a control reagent in a Kentucky Bluegrass pollen diagnostic assay. The methodologies described in this example apply to assays which assist in the diagnoses of allergies to any allergen.

### Production of the Fab2 Fragment

A murine anti-pollen Mab such as Mab 27 described in Ekramoddoullah, etal., J. Immunology (1987) 138:1739-1743 is digested with pepsin as described in Example 1 to yield a Fab2 fragment. Mab27 is specific for an epitope of a Kentucky Bluegrass pollen allergen.

Specifically, Mab 27 is placed into 0.1M citrate, pH 3.5 buffer using a NAP-5 column (Pharmacia). The amount of pepsin (Sigma) added is about 1/30 that of the antibody (w/w) and the reaction is allowed to incubate at 37°. Samples are removed at intervals; an optimal time is determined by examining the progress of the reaction on a Novex electrophoresis gel. Typically, a removal time would occur between around 15 to around 120 minutes, although digestion times of up to 4 hours are possible. The size (approximately 100 KD) and the identity of the Fab2 fragment prepared from the functioning pollen-binding portion of Mab 27 are thereby determined.

For the scaled-up production of the Mab 27 Fab2 fragment, the digestion reaction is performed as optimized and then quenched with 1 M Tris prior to the removal of all non-essential protein fragments by exhaustive dialysis against PBS/2 MM EDTA using a 30K MW membrane.

### Production of the Fc Fragment

Human IgG1 (Fitzgerald catalog #30-AI17) or another polyclonal human IgG or IgE preparation is digested with either papain and ficin enzymes (Sigma) to generate the Fc fragment. The antibody protein is exchanged into a 5OmM Tris, 2 mM EDTA pH 7.0 buffer using NAP-5 (Pharmacia) columns so as to facilitate protein digestion by the enzyme. Either or both of the enzymes are tested at approximately 1/30 (w/w) proportions relative to the antibody. Both enzyme and cysteine are added to the protein and the resultant digestion mixture is allowed to incubate at approximately 37° C.

At timed intervals, aliquots of the reaction are removed and the enzyme reaction in the aliquot is stopped with n-ethylmaleimide. Aliquots are examined on Novex gels to determine the extent of the digestion as evidenced by the fragment sizes produced. Optimum digestion conditions are then assessed. Small portion of the heterogeneous polyclonal human IgG1 preparation could prove resistant to digestion. The resultant

Fc preparation should be predominantly 50 KD in weight, although a single molecular weight will not be assigned to the preparation due to the heterogeneous nature of the native human IgG.

For the scaled-up production of the Fc fragment, the digestion reaction product is purified by passing the reaction product over a protein A column; this removes the Fc region fragment from the remaining digested portions of the IgG1 molecule by allowing the intact Fc to bind to the protein A. The remainder of the digest reaction then flows through the column without binding. After washing the column, the intact Fc fragment is eluted with pH and/or salt conditions and collected. SDS polyacrylamide gel analysis demonstrates that the resultant Fc fragment was correctly recovered.

### Formation of the Humanized Antibody Conjugate

The Kentucky bluegrass binding Mab 27 Fab2 is reduced with mercaptoethylamine to generate Fab molecules which have an available thiol near the hinge region. The Fc fragment derived from human IgG is derivatized with maleimide to facilitate reaction with the available thiol on the Fab.

The Fab2 fragment has a thiol bond between the two cysteines present on each of the two heavy and light chain fragments. Available thiols at locations which are away from the binding site and near the pepsin digestion site are activated by gently reducing the disulfide bond with mercaptoethylamine. A 50 mM MEA Fab2 fragment solution is incubated at 37° C. for 90 minutes. This creates Fab fragments with an intact thiol group and bound heavy and light chains. Excess MEA is removed by desalting the protein mixture on NAP25 columns (Pharmacia) containing a PBS 2mM EDTA reaction buffer.

The Fc fragment is derivatized by addition of a maleimide group to primary amines accessible on the surface of the molecule through reaction with twenty-five fold molar excess SMCC (Pierce) for 25 minutes at room temperature. Excess SMCC is quenched by the addition of glycine and excess SMCC is removed by desalting the protein mixture over a NAP25 column equilibrated in pH 7.4 PBS.

Reduced Fab is added to the activated Fc mixture at a ratio of around 2.4 fold molar excess Fab′: Fc and is allowed to react for around three hours at room temperature. The resultant reaction mixture is stored at between about 2-8° C. for at least 8 hours. Excess maleimides remaining in the reaction mixture are quenched by the addition of around tweleve-fold molar excess MEA.

To separate the resultant Fab:Fc conjugate from the unreacted constituents, the conjugation mixture is chromatographed on a 1.6×60 Superdex 200 column (Pharmacia). A conjugate mixture of approximately 1 mL volume containing 5-10 mg of protein is applied to the column and fractionated by size by running in PBS pH 7.4 buffer at 0.2 ml/min to achieve optimal size separation. Fractions are collected and analyzed by SDS gradient gel (4-20%) for correct size. A single Fab′:Fc conjugate weighs approximately 100 KD, but multimers may be formed in which more than one Fab′ fragment conjugates to a Fc fragment. Additional peaks of greater than 100 KD may therefore be obtained. Pools of conjugate with varying molecular weights are made and tested for performance in an actual assay.

### EXAMPLE 4

### Use of Recombinant Humanized Antibody Conjugate as a Control Reagent

Rubella exposure during pregnancy can have serious consequences during pregnancy. For this reason, many physicians choose to test reproductively competent women for circulating antibodies to Rubella; such antibodies signify past exposure and immunity to the viral infection.

However, it is known that due to immunization programs, Rubella infection is diminishing in the population and there are fewer donors for the Rubella IgG positive sera which is needed as a positive control in the administration of the Rubella serology tests. To circumvent this problem, as well as to provide a uniform supply of this positive control serum, the anti rubella E1 monoclonal antibody, clone R335.6.4 (Bayer) is used as a source of mRNA for the Fab binding region of the murine antibody. The mRNA is treated with reverse transcriptase using any of a number of commercially available kits and the resultant cDNA is ligated into an expression vector which then links the rubella virus epitope E1 binding segment to a human IgG framework. This vector then is used to produce the Bayer humanized antibody in a suitable host, either eukaryotic or prokaryotic. The resultant recombinant Bayer protein molecule has the capability to recognize the Rubella E1 viral epitope and also has the necessary human IgG sequence so that it acts in the Rubella serology assay just as a native, positive patient sample would.

### EXAMPLE 5

### Rubella anti-E1 Humanized IgM Antibody Conjugate

### Production of the Fab2 Fragment

Anti-Rubella E1 Mab (Bayer Clone R335.6.4), deposited under the Budapest Treaty on Jun. 6, 2006 with the American Type Culture Collection, 10801 University Boulevard, Manassas, Va. 20110-2209, (hereinafter “anti-E1”) was used to generate the antigen binding portion of the conjugate. anti-E1 was digested with pepsin to produce a Fab2 fragment from intact IgG1 as follows.

First, digestion time was optimized. Then, anti-E1 was placed into a 0.1 M citrate, pH 3.5 buffer using a NAP-5 column (Pharmacia). The amount of pepsin (Sigma) used to digest anti-E1 was approximately 1/30 of anti-E1 weight and the reaction was incubated at 37° C. Samples were removed at regular intervals and an optimal digestion time of 120 minutes was determined by examining the progress of the digestion on a Novex electrophoresis gel. The Fab2 fragment prepared from the functioning capture portion of anti-E1 weighed approximately 100 KD. See FIG. 1.

To scale-up production of the Fab2 fragment, the digestion reaction was quenched with 1 M Tris, and small digested fragments of the Fc region were thereafter removed by exhaustive dialysis of the reaction against PBS/2 MM EDTA using a 30 K MW membrane.

### Production of the Fc IgM Fragment

Human IgM (Chemicon catalog#AG722) was digested with ficin to generate a humanized antibody conjugate Fc fragment. Since it was not known how the polyclonal human IgM preparation would tolerate digestion, experiments were conducted to identify the best way to prepare the Fc portion of the molecule.

The enzyme ficin (Sigma) was tested with a highly pure human IgM preparation. The antibody protein was exchanged to facilitate digestion into 50 mM Tris, 2 mM EDTA pH 7.0 buffer using NAP-5 (Pharmacia) columns. The enzyme was tested at 1/30 (w/w) proportion relative to the antibody. Both enzyme and cysteine were added to the protein and digested at 37° C. At timed intervals, aliquots of the reaction were removed and the enzyme reaction stopped with n-ethylmaleimide. Aliquots were then examined on Novex gels for examination of the fragment sizes produced. These reaction conditions confirmed that 4.0 hrs was an optimal digestion period, although there remained a small portion of the heterogeneous polyclonal human IgM preparation that was resistant to digestion. See FIG. 2.

The resultant Fc preparation was predominantly 50 KD in weight; no single molecular weight could be determined due to the heterogeneous nature of the native protein mixture.

For the scale-up production of the Fc fragment, the digested, non-reactive medium was purified by passing it over a protein A column. This step served to remove the Fc fragment from the remaining digested portion of the IgM molecule by allowing the intact Fc to bind to the protein A resin, while allowing the remainder of the digest reaction to flow through the column. The intact Fc fragment was then eluted and collected. SDS polyacrylamide gel analysis demonstrated that the resultant Fc fragment pool was recovered. See FIG. 3.

The Fab2 (anti-El) and Fc (IgM) fragments were linked to form a humanized antibody conjugate as follows.

### Formation of the Humanized Antibody Conjugate

The Fab2 and Fc fragments obtained as described above were linked to form a humanized antibody conjugate as follows.

Because the Fab2 fragment was obtained by digestion of anti-E1 in pepsin as described above, the thiol (di-sulfide) bond between the two cysteines on the heavy and light chain fragments of anti-E1 was preserved. (Pepsin digestion cut the anti-E1 Fab2 fragment from intact IgG1 at a location which preserved a thiol (disulfide) bond between the Fab2 fragment heavy and light chains

This thiol (disulfide) bond on the Fab2 fragment was activated by reaction of the Fab2 fragment with 5OmM mercaptoethylamine (MEA) at approximately 37° C. for a period of around 90 minutes. Excess MEA was removed by desalting in a PBS 2 mM EDTA reaction buffer using a NAP25 column (Pharmacia).

The Fc fragment was derivatized by adding a maleimide group to primary amines accessible on the protein surface through reaction of the Fc fragment with SMCC. Specifically, Fc fragment was added to an approximately thirty fold molar excess of SMCC (Pierce) and the resultant reaction mixture was allowed to stand for 30 minutes at room temperature. Excess SMCC was quenched by the addition of glycine to the reaction mixture and excess SMCC was removed by desalting the protein mixture over a NAP25 column equilibrated in pH 7.4 PBS.

The Fab and Fc fragments were combined to form a reaction mixture comprising an approximately 3.7 fold molar excess of Fab to Fc and were allowed to react for approximately sixteen hours at room temperature (overnight) to form humanized anti-E1Rubella antibody conjugate

Next, the humanized Fab:Fc conjugates were recovered using column chromatography. The reaction mixture was chromatographed on a 1.6×60 Superdex 200 column (Pharmacia). A 8.6 milligram conjugate mixture in an approximately 1.5 mL volume was applied to the column and fractionated by size exclusion by elution in a PBS (pH 7.4) buffer at 0.2 ml/min to achieve optimal size separation. Fractions were collected and absorbance at 280 nm of each fraction was read on a spectrophotometer. The absorbance at 280 nm was plotted vs. fraction number. See FIG. 4. Three pools were prepared based on the elution profile, I, II & III. Pool I represents the highest molecular weight conjugate while pool III represents the lowest molecular weight, with pool II intermediate between the two. Pools were concentrated to approx. 1 mg/mL using an ultrafiltration cell equipped with a molecular weight cutoff membrane of 30 kD. The concentration of each pool was measured by A280 using an extinction coefficient of 1.36. As a preservative, Bovine Serum Albumin and Sodium Azide were added to 0.5% and 0.1% respectively. After addition of preservative, the solution was filtered using a 0.2 um filter.

Pools of conjugate were then collected and used as a control reagent in the Bayer ADVIA Centaur® Rubelle IgM assay.

The contents of all published articles, books, reference manuals and abstracts cited herein, are hereby incorporated by reference in their entirety to more fully describe the state of the art to which the invention pertains.

As various changes can be made in the above-described subject matter without departing from the scope and spirit of the present invention, it is intended that all subject matter contained in the above description, or defined in the appended claims, be interpreted as descriptive and illustrative of the present invention. Modifications and variations of the present invention are possible in light of the above teachings.

What is claimed is:

1. A humanized antibody conjugate which comprises a human antibody fragment that is bound by a cross-linking functional group to a non-human antibody fragment, wherein the non-human antibody fragment contains an amino acid sequence which binds to an antigen epitope.

2. The humanized antibody conjugate of claim 1, wherein the human antibody fragment is a Fc fragment of either human IgG or human IgE and the non-human antibody fragment is a Fab2 fragment.

3. The humanized antibody conjugate of claim 1, wherein the human antibody fragment is a Fc fragment of human IgG and the non-human antibody fragment is a Fab2 fragment derived from a murine Mab or rat Mab.

4. The humanized antibody conjugate of claim 1, comprising a Fc fragment of either human IgG or human IgE and a non-human Fab2 antibody fragment, wherein the non-human Fab2 fragment is obtained by digestion of a non-human Mab by an aspartic proteinase.

5. The humanized antibody conjugate of claim 4, wherein:
(a) the non-human Mab is a murine Mab or rat Mab, and (b) a reduced thiol (disulfide) bond on the non-human Fab2 fragment forms a cross-linking functional group with a thiol (disulfide)-binding moiety that has been derivatized onto the Fc fragment.

6. The humanized antibody conjugate of claim 5, wherein:
(a) the non-human Mab is a murine Mab;
(b) the thiol (disulfide) bond on the non-human Fab2 fragment has been reduced by a reducing agent; and
(c) the thiol (disulfide)-binding moiety which has been derivatized onto the Fc fragment is a maleimide group formed by the reaction of the Fc fragment with a maleimide-containing hetero-bifunctional cross-linking agent.

7. The humanized antibody conjugate of claim 6, wherein:
(a) the Fab2 fragment is obtained by digestion of a murine Mab in pepsin;
(b) the Fc fragment is obtained by digestion of human IgG in papain or ficin;
(c) the reducing agent is MEA; and
(d) the hetero-bifunctional cross-linking agent is SMCC.

8. The humanized antibody conjugate of claim 7, wherein:
(a) the murine Mab is NS5; and (b) the Fab2 fragment is obtained by digesting NS5 in pepsin.

9. The humanized antibody conjugate of claim 6, wherein the antigen is a virus or an allergen.

10. The humanized antibody conjugate of claim 9, wherein the antigen is a hepatitis C virus or a pollen.

11. The humanized antibody conjugate of claim 10, wherein the antigen is NS5.

12. A method of making a humanized antibody conjugate which binds to an antigen epitope comprising:
reacting a Fc fragment derived from either human IgG or human IgE with a Fab fragment that is derived from a non-human Mab,
wherein the non-human Mab contains an amino acid sequence that binds to the antigen epitope to form a cross-linking functional group that binds the non-human Fab and human Fc fragments.

13. A method of making a humanized antibody conjugate which binds to an antigen epitope comprising:
(a) digesting a murine or rat Mab in an aspartic proteinase to form a non-human Fab2 fragment which contains an amino acid sequence which binds to the antigen epitope;
(b) digesting a human IgG or IgE in a cysteine (thiol) proteinase to form a human Fc fragment;
(c) reducing a thiol (disulfide) bond on the non-human Fab2 fragment by reacting the non-human Fab2 fragment with a reducing agent;
(d) derivatizing the human Fc fragment with a thiol (disulfide)-binding moiety by reacting the human Fc fragment with a hetero-bifunctional cross-linking agent; and
(e) reacting the reduced non-human Fab2 fragment and derivatized human Fc fragment to form a cross-linking functional group between the human Fc fragment thiol (disulfide)-binding moiety and the non-human Fab2 fragment reduced thiol (disulfide) bond.

14. The method of claim 13, wherein:
(a) a murine Mab is digested in pepsin to form the non-human Fab2 fragment;
(b) a human IgG is digested in papain or ficin to form the human Fc fragment;
(c) the non-human Fab2 fragment is reduced with MEA;
(d) the human Fc fragment is derivatized with a maleimide moiety through reaction with a hetero-bifunctional cross-linking agent; and
(e) the cross-linking functional group is formed between the reduced thiol (disulfide) bond on the non-human Fab2 fragment and the human Fc fragment maleimide moiety.

15. The method of claim 14, wherein the non-human Fab2 fragment and the human Fc fragment are reacted in a weight ratio of from about 1.5:1 to about 5:1.

16. The method of claim 15, wherein the non-human Fab2 fragments and the human Fc fragments are reacted in a weight ratio of from about 2:1 to about 3:1.

17. The method of claim 15, wherein the human IgG is digested in papain and the hetero-bifunctional cross-linking agent is SMCC.

18. The method of claim 17, wherein the murine Mab binds to an epitope that is the same as or equivalent to the epitope of NS5.

19. An analytical kit comprising a control reagent comprising a humanized antibody conjugate of claim 1.

20. An analytical kit comprising a control reagent comprising a humanized antibody conjugate of claim 6.

21. An analytical kit comprising a control reagent comprising a humanized antibody conjugate of claim 9.

22. An analytical assay control reagent comprising a humanized antibody conjugate of claim 1.

23. An analytical assay control reagent comprising a humanized antibody conjugate of claim 6.

24. An analytical assay control reagent comprising a humanized antibody conjugate of claim 9.

25. A method for evaluating the performance of an analytical assay comprising using a control reagent comprising a humanized antibody conjugate of claim 1.

26. A method for evaluating the performance of an analytical assay comprising using a control reagent comprising a humanized antibody conjugate of claim 6.

27. A method for evaluating the performance of an analytical assay comprising using a control reagent comprising a humanized antibody conjugate of claim 9.

28. A analytical kit comprising a control reagent, wherein the control reagent is a recombinantly-derived humanized antibody conjugate.

29. A method for evaluating the performance of an analytical assay by using a recombinantly-derived humanized antibody conjugate.

30. The method of claim 29, further comprising the step of observing of a positive signal.
